(19) United States
(12) Patent Application Publication
(10) Pub. No.: US 2019/0307828 A1
(43) Pub. Date: Oct. 10, 2019

(54) DIETARY VEGAN SUPPLEMENT FOR TREATING JOINT PAIN

(71) Applicants: Ashish Upadhyay, Irvine, CA (US); Dhwani Upadhyay, Irvine, CA (US)

(72) Inventors: Ashish Upadhyay, Irvine, CA (US); Dhwani Upadhyay, Irvine, CA (US)

(21) Appl. No.: 15/945,036

(22) Filed: Apr. 4, 2018

(51) Int. Cl. A61K 36/9066; A61P 19/02; A61K 31/355; A61K 36/82; A61K 36/9068; A61K 36/63; A61K 31/7008; A61K 36/55; A61K 36/67; A61K 36/53; A61K 31/593; A61K 33/10; A23L 33/105; A23L 33/155; A23L 33/16

(52) U.S. Cl. CPC A61K 36/9066; A23V 2002/00; A61K 31/355; A61K 36/82; A61K 36/9068; A61K 36/63; A61K 31/7008; A61K 36/55; A61K 36/67; A61K 36/53; A61K 31/593; A61K 33/10; A23L 33/105; A23L 33/155; A23L 33/16; A61P 19/02

(57) ABSTRACT

The present disclosure relates to a dietary vegan supplement for treating joint pain in human comprising of natural ingredients as its constituents. The composition for the dietary vegan supplement for treating joint pain in human comprises of natural vitamin E (d-alpha-tocopherol), turmeric root extract (standardized to 95% curcuminoids), a plant material (devils claw) providing a rich source of harpagosides, calcium carbonate powder, green tea extract comprising standardized 90% polyphenols, 80% catechins, ginger, olive extract (olive oil powder), glucosamine HCL, flaxseed or flaxseed extracts thereof, and black pepper extract standardized to contain 95% piperine. In addition, the compositions provided herein can contain other ingredients such as vitamin D3 (cholecalciferol).

### FIELD OF THE INVENTION

The present disclosure relates to the field of dietary supplements and particularly to a dietary vegan supplement for treating joint pain in human.

### BACKGROUND

Joint pain is a common complaint now days. Typically, joint pain refers to discomfort, aches, stiffness, and soreness in any of the body's joints. Joint pain may be experienced at low back, shoulder, knees, ankles or many other parts of the body. The joint pain mainly arises when ligaments, cartilage, and bones within the join are affected. The joint pain may be caused due to injury or illness (such as bone infections, cancer etc). The most common cause for the joint pain now days are seen in the form of Arthritis. Arthritis causes joint pain and is most common in adults of age 40 and above. Many doctors consider treatment of the joint pain from arthritis to be quite impossible despite there exists lots of medications (such as non-steroidal anti-inflammatory drugs (NSAIDs), example Cox-2 inhibitors (celcoxib)) to relive the joint pain arising out of the Arthritis. Although such anti-inflammatory drugs may relieve from joint pain, such drugs can lead to several side effects such as liver or kidney damage. In addition, long-term use of such drugs can lead to reduced effectiveness.

Further, lots of drugs are available in the marketplaces and there are millions of people who rely on taking pain-killers such as Aspirin, Ibuprofen, or naproxen sodium frequently to help manage their ongoing joint or bone pains. However, long run exposure to such over the counter medications can cause several health issues, which is undesirable. Also, it is known fact that most of the medications or drugs involve uses of animal products in their composition or during their manufacturing. There is huge population out there suffering from joint pain that only consider natural treatment for such joint pains and are not open to take up drugs that make use of animal products and may cause various side effects to their health.

Attempts are made in the past and several compositions are proposed for relieving the joint pain. For example: U.S. Pat. No. 4,592,912 discloses about an aqueous extract comprising which includes from about 92 to 99.75 parts by weight sterile water, from about 0.25 to 6.0 parts by weight cayenne (Capsicum frutescens), from about 0.02-1.0 parts by weight slippery elm powder, from about 0.02-1.0 parts by weight myrrh gum granules or powder and a preservative mixture that is included to insure that the extract remains stable over a prolonged period of time comprising from about 0.3-0.5 parts by weight benzoic acid and from about 0.1-0.3 parts by weight tetrasodium EDTA.

Further, there are compositions in the form of supplements or tablets presented in the past which made uses of natural ingredients, for example U.S. Pat. No. 5,8852,653 describes about a capsule or tablet for treating pain in a human in need thereof consisting of therapeutically effective amounts of turmeric extract, Boswellia extract, ginger extract, holy basil extract, rosemary extract, white willow extract, alpha lipoic acid and trolamine salicylate, U.S. Pat Appl No. 20040185035 discloses a composition for a dietary supplements. According to patent application, the compositions can be used to reduce pain, inflammation, stiffness, and/or discomfort associated with inflammatory conditions such as arthritis. According to the patent application the dietary supplement comprises of an aminosaccharide, a ginger component, and an enzyme.

Despite attempts are made to provide variety of compositions in the form of aqueous extracts or supplements that make use of natural ingredients fully or partially, there still remains a need for an improved and effective composition that can reduce the joint pain within a short time period and comprises of vegan natural ingredients.

### BRIEF SUMMARY

Disclosed is an invention that provides a natural composition for dietary vegan supplement for reducing joint pain in human.

The proposed composition of the invention provide people suffering from joint pain with a treatment that can produce detectable benefits within a very short time period (e.g., within a few months).

The proposed composition provided herein can help people live healthier, more active lives by reducing joint pain naturally using vegan supplements.

According to an aspect, the composition for a dietary vegan supplement for treating joint pain in human in a therapeutically effective amounts of natural vitamin E (d-alpha-tocopherol), turmeric root extract (standardized to 95% curcuminoids), a plant material (devils claw) providing a rich source of harpagosides, calcium carbonate powder, green tea extract comprising standardized 90% polyphenols, 80% catechins, ginger, olive extract (olive oil powder), glucosamine HCL, flaxseed or flaxseed extracts thereof, and black pepper extract standardized to contain 95% piperine. In addition, the compositions provided herein can contain other ingredients such as vitamin D3 (cholecalciferol).

According to another aspect, a method for maintaining healthier lives (more active lives) in human, by reducing joint pain naturally using vegan supplements comprising the step of providing to a subject, a composition comprising in an effective amount natural vitamin E (d-alpha-tocopherol), turmeric root extract (standardized to 95% curcuminoids), a plant material (devils claw) providing a rich source of harpagosides, calcium carbonate powder, green tea extract comprising standardized 90% polyphenols, 80% catechins, ginger, olive extract (olive oil powder), glucosamine HCL, flaxseed or flaxseed extracts thereof, black pepper extract standardized to contain 95% piperine, and vitamin D3 (of cholecalciferol).

These and other features and advantages of the invention will become apparent from the detailed description below, in light of the accompanying drawings.

### DETAILED DESCRIPTION

Disclosed herein is a dietary vegan supplement for treating joint pain in human comprising of, in therapeutically effective amounts, natural ingredients. The composition for the dietary vegan supplement for treating joint pain in the human comprises of natural vitamin E (d-alpha-tocopherol), turmeric root extract (standardized to 95% curcuminoids), a plant material (devils claw) providing a rich source of harpagosides, calcium carbonate powder, green tea extract comprising standardized 90% polyphenols, 80% catechins, ginger, olive extract (olive oil powder), glucosamine HCL, flaxseed or flaxseed extracts thereof, and black pepper extract standardized to contain 95% piperine. In addition, the compositions provided herein can contain other ingredients such as vitamin D3 (cholecalciferol).

Descriptions of some key ingredients/constituents contained in the dietary vegan supplement of the present invention are provided in the following paragraphs:

Natural vitamin E (d-alpha-tocopherol): Vitamin E (as d-alpha tocopheryl or mixed tocopheryls) is a powerful antioxidant that is vital in maintaining good health. Supplementation of vitamin E is important. Vitamin E is actually a family of different compounds. Although d-alpha tocopherol is the most active antioxidant, increasing research indicates that other tocopherols, especially gamma-tocopherol, are also beneficial. The vitamin E used in the dietary vegan supplement (d-alpha tocopherol) is absorbed much better than any other synthetic form (dl-alpha tocopherol).

Vitamin D3 (of cholecalciferol): Vitamin D3 in this form is considerably more active than other forms such as vitamin D2 (ergocalciferol) and hence it is more effectively used in the proposed composition.

Turmeric Root Extract (standardized to 95% curcuminoids): Turmeric Root Extract (containing 95% curcuminoids) is also known as curcumin, and is a spice with various advantageous qualities. In addition to being an effective anti-inflammatory herb, turmeric has potential antioxidant property.

Green tea extract comprising standardized 90% polyphenols, 80% catechins: Green tea extracts have been shown to be potent inhibitors of inflammation and hence finds a useful way in the proposed composition.

Ginger: Ginger is certified organic ginger according to the invention.

Olive extract (olive oil powder): one or more extracts of olive (e.g., olive oil, olive powder, etc.), the extracts, may or may not be in the form of oil (or derived from an oil), may be obtained from any part of the olive tree including the leaves, stems, flowers, fruits, bark, and roots.

Glucosamine HCL: glucosamine HCL in proposed composition is aimed at improving cartilage health. It is believed that glucosamine, or similar products, provide one or more of the biochemical components which strengthens the cartilage in human.

Black pepper extract standardized to contain 95% piperine: Black pepper is a natural herb which is generally well recognized as safe for its intended use as spice, seasoning, or flavoring. Black pepper spice is obtained from peppercorns from fruits of Piper nigrum and is used widely throughout the world. Black pepper has also been traditionally used in the Indian system of medicine ‘Ayurveda’ for the treatment of many ailments. Piperine (1-peperoylpiperidine), the alkaloid that is the active ingredient of black pepper, has various properties and uses, including flavoring, insecticide activity, antidiarrhoeal activity, anti-inflammatory activity, ability to stimulate the release of epinephrine, ability to elevate levels of circulating thyroid hormones, antioxidant function. The bioenhancing dose of piperine (95% piperine) as used in the present invention enhances the use of Black pepper extract for treating the joint pain.

Based upon the above-identified description of the ingredients for the dietary vegan supplement for treating joint pain in the human, the preferred formulation for the supplement is listed in Table 1 below. Although according to the preferred embodiment, the supplement of the present invention is made available in capsule form but those skilled in the art will appreciate in view of this disclosure that the supplement of the present invention may also be provided in many other forms, including pill or tablet, powder etc., without departing from the scope of this disclosure.

TABLE 1
INGRIDIENTS

| | |
|---|---|
| Natural vitamin E (d-alpha-tocopherol) | 26 mg |
| Vitamin D3 (cholecalciferol) | 0.02 mg |
| Turmeric root extract (standardized to 95% curcuminoids) | 150 mg |
| A plant material (devils claw) | 200 mg |
| Calcium carbonate powder | 499 mg |
| Green tea extract (standardized 90% polyphenols, | 200 mg |
| 80% catechins) | |
| Ginger (organic) | 100 mg |
| Olive extract (olive oil powder) | 100 mg |
| Glucosamine HCL (vegan) | 100 mg |
| Flaxseed or flaxseed extracts thereof | 50 mg |
| Black pepper extract (standardized to contain 95% piperine) | 5 mg |

The allowable dosage range for the dietary vegan supplement that provides relieve from joint pain may depend upon the severity of subject/human suffering from the joint pain as well as their age. However, the ideal dose for the adults is 2 tablets or capsules/day.

Evaluations of the Composition

Case 1: A 67 year old 100% vegetarian male, having knee pain, the subject was suggested surgery and he wanted to avoid surgery. He took the proposed dietary supplement daily with 2 capsules/day. After 3 months, the subject felt considerable positive change in his keen pain. The subject is able to now climb stairs without much trouble.

Case 2: A 35 years old female, who loves long trail hike, often gets joint pains after long hike. After consuming the proposed joint supplement for 2 months (2 capsules/day) felt significant relief in her joints pain after her long hikes.

Case 3: A 48 years old male. The subject had joint pain in his tennis elbow due to excessive workouts. After consuming proposed joint supplement for 4 months (2 capsules/day), the subject's pain in his tennis elbow is significantly reduced.

Case 4: A 56 year old female, overweigh, had developed joint pain due to weight related problems. After consuming 2 capsules/day of the proposed joint supplement, the subject had reduced joint pain after 5 months.

Case 5: A 31 years old male, marathon runner, wanted to reduce joint pain he developed after wrong runs. After consuming proposed joint pain supplement for over 2 months period felt significant relief in his joint pain.

The following procedure is performed to make a capsule out of the proposed composition for the supplement.

Blending: The gathering of raw materials/ingredients and weighing them into appropriate container for preblending.

Screening: All ingredients are weighed and prescreened through a clean stainless steel screen, consisting with a 150 mesh.

Conveying: All ingredients are then conveyed or added by hand to a drum style mixer or a Nauta mixer. Batch is then mixed for 20 minutes. Once mixed, materials are transferred to 55 gallon drums for movement to the encapsulating machines.

Encapsulating: Powder is received from the Blending/Screening area and is then ready for the encapsulating process. Encapsulating machines are either TruStar or JTJ-A Ruian Vertical Semi Automatic fillers or Schaefer or Shionogi Horizontal fillers commercially available.

Dedusting/Quality Inspection: Once encapsulating is complete, capsules are filled into 5 gallon pails and taken to the inspection table. The dedusting and inspection process then begins. Capsules are hand-inspected for defects and improper sealing.

Polishing/Finishing: Dedusted capsules are then moved to the Polishing Machine (rotating brush style) for finishing.

Bottling/Packaging: Capsules can be bottled using either a Phoenix Disc Counter Machine or a King Digital Capsule Counting Machine commercially available. Packaging consists of each individual bottle being packed with cotton, lids attached, shrink bands attached, and final sealing with hand held heat gun. Once sealed, bottles are packaged into card board boxes, sealed and prepared for shipping.

Labeling and coding : Each bottle used in the process is labeled individually by a JDA labeling machine. Bottles are conveyed, while spool of labels on the machine adds one label per bottle. Bottles are stamped individually with a commercially available Cintronix coding machine. Each bottle may receive a manufacturing date, lot number and ‘best before’ date etc, based on order/production schedule.

The supplement or composition according to the present invention is preferably encapsulated by a water-soluble capsule. Capsules are preferred to deliver the supplement because capsules are generally better absorbed than tablets. Although properly manufactured tablets should dissolve in the presence of stomach acids and could be used to deliver the supplement of the present invention, people with low-acidity or poor digestion may not be able to break down the tablet in their internal system. Another reason for using capsules is that tablets are pressed using pressure and high heat, which may destroy active ingredients and compromise potency of the supplement. However the proposed supplements may still be manufactured in the form of tablets. The production of the supplement in the form of tablets may require additional steps in the manufacturing process compared to capsule production and labeling steps described above. The tablets are formed from raw ingredients which are compressed in a tablet press. Typical tablet production procedure is as under:

Blending: Blending: The gathering of raw materials/ingredients and weighing them into appropriate container for preblending.

Screening: All ingredients are weighed and prescreened through a clean 150 mesh stainless steel screen.

Conveying: Ingredients are then conveyed or added by hand to a drum style mixer or a Nauta mixer commercially available. Batch is mixed for 20 minutes. Once mixed, materials are transferred to 55 gallon drums for movement to the tableting machine known in the art.

Tablet Production: Powder is received from the Blending/Screening area and is ready for the tableting process. Tableting machines are either a Stokes D3 Tablet Press with a Reeves Variable Speed Drive, or small Stokes Tablet Press.

Dedusting/Quality Inspection: Once tableting is complete, tablets are filled into 5 gallon pails and taken to the inspection table. The dedusting and inspection process begins. Stainless steel screen size of 0.125″ opening is used for proper separation. Tablets are hand-inspected for defects.

Sealing/Finishing: Dedusted/inspected tablets are then moved to the sealing area, a pharmaceutical glaze is added to the tablet to seal the product and give it a smooth surface for ingesting. Sealing may/may not be performed based on product. Sealer is added to tablets by dipping tablets in a glaze solution. Tablets are then tumbled in a Pan Coater known in the art for a finished product before moving to the bottling/packaging area.

Bottling/Packaging: Tablets can be bottled using either a Phoenix Disc Counter Machine, a King Digital Capsule/Tablet Counting Machine known in art, or depending if the tablets are large, can be bottled by hand. Packaging consists of each individual bottle being packed with cotton, lids attached, shrink bands attached, and final sealing with hand held heat gun. Once sealed, bottles are packaged into card board boxes, sealed and prepared for shipping.

Labeling/Coding: Each bottle used in the process is labeled individually by a JDA labeling machine known in the art. Bottles are conveyed, while spool of labels on the machine adds one label per bottle. Bottles are coded individually with a Cintronix coding machine known in the art. Each bottle receives a manufacturing date, lot number and ‘best before’ date, based on order/production schedule.

In the description and claims of this specification the word “comprise” and variations of that word, such as “comprises” and “comprising” are not intended to exclude other features, additives, components, integers or steps but rather, unless otherwise stated explicitly, the scope of these words should be construed broadly such that they have an inclusive meaning rather than an exclusive one.

Although the therapeutic compositions of the invention have been described in the present disclosure by way of illustrative examples, it is to be understood that the invention is not limited thereto and that variations can be made as known by those skilled in the art without departing from the teachings of the invention defined by the appended claims.

We claim:

1. A dietary vegan supplement for treating joint pain in a human comprising essentially of therapeutically effective amounts of natural vitamin E (d-alpha-tocopherol), turmeric root extract (standardized to 95% curcuminoids), a plant material providing a rich source of harpagosides, calcium carbonate powder, green tea extract comprising standardized 90% polyphenols, 80% catechins, ginger, olive extract (olive oil powder), glucosamine HCL, flaxseed or flaxseed extracts thereof, and black pepper extract standardized to contain 95% piperine.

2. The supplement of claim 1, wherein the plant material is devils claw.

3. The supplement of claim 1 further comprising vitamin D3 (of cholecalciferol).

4. The supplement of claim 1, wherein the ginger is an organic ginger or ginger extract.

5. A method for reducing joint pain in human, the method comprising administering a dietary supplement, to the human, in an amount effective to reduce the joint pain, wherein the dietary supplement comprises of natural vitamin E (d-alpha-tocopherol), vitamin D3 (of cholecalciferol), turmeric root extract (standardized to 95% curcuminoids), a plant material providing a rich source of harpagosides, calcium carbonate powder, green tea extract comprising standardized 90% polyphenols, 80% catechins, ginger, olive extract (olive oil powder), glucosamine HCL, flaxseed or flaxseed extracts thereof, and black pepper extract standardized to contain 95% piperine.

6. The method of claim 5, wherein the plant material is devils claw.

7. The method of claim 5, wherein the ginger is an organic ginger or ginger extract.

8. A dietary vegan supplement for treating joint pain in a human comprising at least about 26 mg of natural vitamin E (d-alpha-tocopherol), at least about 150 mg turmeric root extract (standardized to 95% curcuminoids), at least about 200 mg devils claw, at least about 499 mg calcium carbonate powder, at least about 200 mg green tea extract comprising standardized 90% polyphenols, 80% catechins, at least about 100 mg ginger, at least about 100 mg olive extract (olive oil powder), at least about 100 mg glucosamine HCL, at least about 50 mg flaxseed or flaxseed extracts thereof, and at least about 5 mg black pepper extract standardized to contain 95% piperine.

9. The supplement of claim 8 further comprises at least about 0.02 mg of vitamin D3 (of cholecalciferol).

10. The supplement of claim 8, wherein the ginger is an organic ginger or ginger extract.
